(19) United States
(12) Patent Application Publication
(10) Pub. No.: US 2009/0253600 A1
(43) Pub. Date: Oct. 8, 2009

(54) Underarm cleanser

(76) Inventors: Anahid Arsharuni, Tujunga, CA (US); Morton A. Westman, Oak Brook, IL (US)

Correspondence Address: DYKEMA GOSSETT PLLC, FRANKLIN SQUARE, THIRD FLOOR WEST, 1300 I STREET, NW, WASHINGTON, DC, 20005, US

(21) Appl. No.: 12/457,511

(22) Filed: Jun. 12, 2009

Related U.S. Application Data
(62) Division of application No. 11/984,066, filed on Nov. 13, 2007.
(60) Provisional application No. 60/858,367, filed on Nov. 13, 2006.

(51) Int. Cl. A61K 8/362

(52) U.S. Cl. 510/137

(57) ABSTRACT

A cleanser composition for removal of antiperspirant residue that includes insoluble metal salts comprises a surfactant, 0.01 to 2.5 wt % of chelating agent, and a diluent.

### CROSS-REFERENCE TO RELATED APPLICATION

This application is based on U.S. Provisional Application No. 60/858,367, filed Nov. 13, 2006, the priority of which is hereby claimed.

### BACKGROUND OF THE INVENTION

The present invention relates to body cleansers (body washes).

Typical body washes and cleansers are used to remove sebum, dirt and various residues from a user's skin when, for example, taking a shower. However, although known washes and cleansers are effective for general cleaning, they are not effective in removing underarm residues primarily consisting of antiperspirant residue and including metallic antiperspirant agents (primarily aluminum salts). Such residues form in the arm pit area, leading to a feeling of dirtiness in that area that persists through current cleansing techniques. Such residues create the tactile sensation of drag or friction when the hand is passed through the underarm during cleansing and may leave the user feeling unclean and discontent with their cleansing regimen. Such residues also discolor (primarily yellow) garments that come into direct contact with these areas, even after being freshly cleaned.

The present inventors have discovered a cleansing composition which is effective in removing underarm residues primarily consisting of antiperspirant residue and including metallic residues of antiperspirants from a user's skin, and in particular in their underarm areas.

### SUMMARY OF THE INVENTION

According to this invention, a chelating agent is combined with cleaning surfactants so as to provide a composition which will remove residues primarily consisting of antiperspirant residue and including insoluble antiperspirant metal salts from a user's skin, and in particular from their underarm areas.

Although chelating agents have been previously used in personal care products for various purposes for example in hair dye formulations to trap unwanted metals and thus prevent premature oxidation of the dyestuffs therein and distortion of their intended color, and in other products to increase the effectiveness of microbiological preservatives, their use in body cleaners to enhance the cleansing process and to specifically assist the removal of metallic salt is not previously known. Example formulations of the inventive composition are found in Table 1.

TABLE 1

| | INGREDIENT -- | | INGREDIENT -- | FORMULA | CONC. RANGE | | | |
|---|---|---|---|---|---|---|---|---|
| | TRADE NAME | SOURCE | INCI NAME | 071806-01 | Broad | Pref. | PURPOSE | ALTERNATES |
| 1 | Deionized Water | Local | Water | 60.880 | | | Diluent, solvent | None |
| 2 | Natrosol 250 | (1) | Hydroxyethyl- | 1.750 | 0.1-5.0 | 0.2-2.0 | Thickening, provide | Numerous including: |
| | HHR CS | | cellulose | | | | lubricity during | various substituted |
| | | | | | | | application | Cellulosics; Carbomer |
| 3 | Plantaren 2000 | (2) | Decyl | 10.000 | 1.0-40 | 5.0-20 | Mild nonionic | Anionic & nonionic |
| | (97% active) | | Glucoside | | | | surfactant for | surfactants (but none |
| | | | | | | | cleansing & formula | quite as appropriate) |
| | | | | | | | homogeneity | including: Sodium |
| | | | | | | | | Lauryl Sulfate; |
| | | | | | | | | Ammonium Laureth Sulfate |
| 4 | Crodasinic LS-30 | (3) | Sodium Lauroyl | 20.000 | 1.0-60 | 5.0-30 | Mild anionic | Numerous anionic |
| | (30% active) | | Sarcosinate | | | | surfactant for | surfactants (but none |
| | | | | | | | cleansing & formula | quite as appropriate) |
| | | | | | | | homogeneity | including: TEA Lauryl |
| | | | | | | | | Sulfate; Ammonium Cocoyl |
| | | | | | | | | Isethionate |
| 5 | Monamid CMA | (4) | Cocamide MEA | 3.000 | 0.5-20 | 2.0-10 | Lather thickener; skin | Numerous amides and amine |
| | | | | | | | conditioner | oxides including: PPG-1 |
| | | | | | | | | Hydroxyethyl Caprylamide; |
| | | | | | | | | Lauramide DEA. |
| 6 | Monateric CAB-LC | (4) | Cocamido- | 2.000 | 0.5-30 | 2.0-15 | Mild amphoteric | Numerous amphoteric |
| | (35% active) | | propylbetaine | | | | surfactant for lather | surfactants inducing: Oleyl |
| | | | | | | | thickening & skin | Betaine; Behenyl Betaine; |
| | | | | | | | conditioning | |
| 7 | Stepan EGMS | (5) | Ethylene Glycol | 1.500 | 0.1-6.0 | 0.5-3.0 | Opacifier & pearlizing | Ethylene glycol distearate; |
| | | | Monostearate | | | | agent | latex emulsions |
| 8 | Versene NA | (6) | Disodium EDTA | 0.100 | 0.01-2.5 | 0.05-1.0 | Chelating agent for | Other forms of EDTA |
| | (Disodium | | | | | | removal of metal salts | (Ethylenediamine |
| | EDTA) | | | | | | & preservation aid | Tetraacetic Acid) |
| 9 | Aloe Vera | (7) | Aloe Barbadensis | 0.010 | 0.001-10 | 0.01-3.0 | Natural ingredient for | Numerous including: |
| | Powder 200:1 | | Leaf Juice | | | | skin soothing. | Althaea Officinalis; |
| | Extract | | Powder | | | | | Comfrey; Allantoin; |
| 10 | Hydroglycolic | (7) | Anthemis Nobilis | 0.010 | 0.001-10 | 0.01-3.0 | | Calendula Officinalis; |
| | Extract of | | Flower Extract | | | | | Nasturtium Officinale |
| | Roman Camomile | | | | | | | Extract |
| 11 | Glydant | (8) | DMDM Hydantoin | 0.500 | 0.01-3.0 | 0.2-2.0 | Microbiological | Numerous including: |
| | | | | | | | preservative | para-bens, |
| | | | | | | | | Imidazolidinyl Urea; |
| | | | | | | | | Methylchloroisothiazolinone |
| 12 | BA 24234 | (9) | Fragrance | 0.250 | 0.05-3.0 | 0.05-2.0 | Fragrance | Numerous |
| | | | | 100.000 | | | | |

Viscosity (25 C., RVT, Sp6; 50 RPM) = approx. 11,200 cps; pH = 7.2 +/− 0.3 (25 C.)
Manufacture: Slowly sprinkle Natrosol to water at RT with rapid mixing. When fully suspended add 3 and 4. Then reduce stirring speed to slow- moderate and Heat to 72-77 C. When free of clumps, fish eyes etc., add 5-8, in order. Allow to cool and add remainder of ingredients below 35° C.
(1) Aqualon Division of Hercules Inc
(2) Cognis Corporation, Care Chemicals
(3) Croda Inc.
(4) Uniqema
(5) Stepan Company
(6) Dow Chemical
(7) International Sourcing Incorporated
(8) Lonza, Inc.
(9) Belle Aire Fragrances

1-8. (canceled)

9. A method of removing antiperspirant residues containing soluble metal salts from a user's skin, comprising the steps of:
a. providing a composition which comprises a surfactant, an effective amount of a chelating agent, and a diluent,
b. applying the composition provided in step (a) to the antiperspirant residues containing insoluble metal salts on the skin of the user, and
c. removing the combined composition and antiperspirant residues of step (b) from the user's skin.

10. The method of claim 9, wherein said antiperspirant residues containing insoluble metal salts are in the user's armpit area.

11. The method of claim 10, wherein step (c) comprises rinsing the composition and the antiperspirant residues from the user's armpit area.
